(19) United States
(12) Patent Application Publication
(10) Pub. No.: US 2024/0157386 A1
(43) Pub. Date: May 16, 2024

(54) MOISTURE CONTROL APPARATUS

(71) Applicants: Aadi GANNAVARAM, Potomac, MD (US); Sree Samhita Gundimeda, Gandhinagar (IN)

(72) Inventors: Aadi GANNAVARAM, Potomac, MD (US); Sree Samhita Gundimeda, Gandhinagar (IN)

(21) Appl. No.: 18/507,022

(22) Filed: Nov. 11, 2023

Related U.S. Application Data
(60) Provisional application No. 63/424,497, filed on Nov. 11, 2022.

(51) Int. Cl. B05B 12/00; A61L 2/10; A61L 2/26; B05B 12/02; B05B 12/08; B05B 12/12; G08B 21/20

(52) U.S. Cl. CPC B05B 12/004; A61L 2/10; A61L 2/26; B05B 12/02; B05B 12/084; B05B 12/12; A61L 2202/11; G08B 21/20

(57) ABSTRACT

An embodiment relates to an apparatus comprising a spray, an actuator, a water compartment, a memory, and a processor. The processor is communicatively coupled to the memory and wherein the data processor is operable to actuate the actuator and activate the spray. The apparatus is operable to control humidity of a reed stored in a storage case.

### RELATED APPLICATIONS

This application claims the benefit under 35 U.S.C § 119 of U.S. Provisional Application No. 63/424,497, filed on Nov. 11, 2022, and U.S. Provisional Application No. 63/414,177, filed on Nov. 10, 2022, which are hereby incorporated by reference in their entirety.

### FIELD OF THE INVENTION

This invention relates to an apparatus to control moisture. The invention more particularly relates to a device to control moisture of a reed in a reed storage apparatus.

### BACKGROUND

In this section the prior art relevant to the field is cited.

“An integrated reed protection and storage system is provided. The system includes a controlled solution container. The system also includes a sponge material positionable within the container. Further, the system includes at least one reed holder positionable around the sponge material. Additionally, the system includes at least one reed, the at least one reed positionable on the at least one reed holder. The at least one reed is configured to contact a surface of the sponge material.” [Source: Integrated reed protection and storage methods and systems; published as U.S. Ser. No. 10/460,706B2 on Oct. 29, 2019]

“An automatic plant watering apparatus having a cavity for receiving a standard houseplant pot, a reservoir for holding water, a pump for removing water from the reservoir, a host for delivering water from the pump to a plant positioned in the cavity. The pump is operated by a microcontroller that is programmed to measure a first time period during which the pump is not operated, and a second time period during which the pump is operated. According, the apparatus can be programmed to automatically water a house plant at predetermined intervals, as well as deliver a predetermined amount of water.” [Source: Plant watering system; published as U.S. Pat. No. 8,408,229B2 on Apr. 2, 2013]

“Embodiments of the present invention are generally directed to systems and methods for remote irrigation control. An exemplary embodiment of the present invention provides a remote irrigation control system having a rain sensor enabled to detect the amount of rain received in a first rain area, and a first local transceiver in communication with the rain sensor, the first local transceiver configured to receive information from the rain sensor and transmit the information to a gateway, the first local transceiver also configured to receive information from the gateway. The gateway is connected to a wide area network and configured to receive information from the first local transceiver and transmit the information to the wide area network, the gateway also configured to receive information from the wide area network and transmit the information to the first local transceiver. Other aspects, features, and embodiments are also claimed and described.” [Source: Systems and methods for remote irrigation control; published as U.S. Pat. No. 8,193,930B2 on Jun. 5, 2012]

“A woodwind reed storage and maintenance device conveniently fits directly onto the reed bearing end of a musical instrument. The storage and maintenance device includes a reed storage space, humidity control device space, and a humidity gauge. The storage and maintenance device is used to maintain a reed while still located on the musical instrument at a specifically chosen constant humidity level for optimal performing condition and maximum reed life. The storage and maintenance device protects the reed from physical damage and from damage due to changing humidity levels in the ambient surroundings. The storage and maintenance device accurately measures and displays temperature and humidity information corresponding to the reed storage space inside the device.” [Source: Humidity controlled musical instrument reed cap; published as U.S. Ser. No. 10/832,641B2 on Nov. 10, 2020]

“A storage and maintenance apparatus for a musical instrument such as a woodwind or bagpipes comprises a housing 102 for surrounding a reed, the housing including a humidity control device such as silica gel, a sponge, or a two-way humidity control pouch (Boveda) and an electronic digital hygrometer and/or thermometer 100 preferably with an LCD display. Preferably the housing has a body 102 and cap 100 at one end and an opening at the other end, the cap containing the hygrometer and/or thermometer. The body being cylindrical in shape with an internal humidity control device holder 108 for creating a reed storage area 104 and separating the humidity control device from contacting the reed (not shown). The body is preferably made from silicone material and sized to create a snug fit with a chanter or musical instrument when fitted. A lock screw 106 may be provided for securing the apparatus to the musical instrument.” [Source: Musical instrument read storage and maintenance device with humidity control and digital hygrometer and thermometer; published as GB2565207A on Jun. 26, 2019]

There is a need to control moisture in reeds in a reed storage apparatus. There is a need for a device that removes the excess moisture from the reeds and maintains an optimum moisture level to prevent the reed from any damage due to the moisture.

### SUMMARY

An embodiment relates to a device comprising a suction channel, a fan, and a desiccant. The device is a handheld device operable to remove moisture from a musical instrument.

In an embodiment, the fan is connected to a proximal end of the suction channel.

In an embodiment, the device further comprises a recess connected to a tubing.

In an embodiment, the device is attachable to a woodwind instrument through the recess to remove moisture from a reed of the woodwind instrument.

In an embodiment, the device is attachable to a mouthpiece of the woodwind instrument.

In an embodiment, the recess is made of flexible material that fixes the device on the mouthpiece of varying size.

In an embodiment, a compartment at a distal end of the suction channel comprises the desiccant.

In an embodiment, the desiccant comprises at least one of a silica gel and a humidity control pouch.

In an embodiment, the fan is operated using a switch.

In an embodiment, the fan removes the moisture by suction of the moisture into the compartment comprising the desiccant.

In an embodiment, the fan is a suction fan.

In an embodiment, a fan blade designed at an angle to pull out the moisture from the woodwind instrument.

An embodiment relates to a device comprising a suction channel, a suction connector, a suction tubing, and a source of a suction force. The device is operable to remove moisture from a reed.

In an embodiment, the device is an elongated device comprising a recess at a proximal end of the device operable to attach the device on a mouthpiece of a woodwind instrument.

In an embodiment, wherein the suction channel is inside the device.

In an embodiment, the suction connector is connected at a distal end of the device.

In an embodiment, the suction tubing is connected between one end of the suction channel and the recess.

In an embodiment, the source of the suction force is attached to the suction connector to provide the suction force.

In an embodiment, the source of the suction force comprises at least one of a suction pump, a vacuum pump, an air pressure pump, and a fan.

In an embodiment, the suction force is adjusted to dry the reed or the woodwind instrument.

In an embodiment, the device comprises a desiccant.

In an embodiment, the desiccant comprises at least one of a silica gel and a humidity control pouch.

In an embodiment, the device is connected to a music stand.

In an embodiment, the device is operated by a switch.

In an embodiment, the device comprises a humidity sensor to measure a value of humidity of the reed.

In an embodiment, the device comprises a processor and a memory; and wherein the processor is operable to operate the device.

In an embodiment, the processor is operable to receive the value of humidity, compare the value of humidity with a pre-set level of humidity. The device maintains the pre-set level of humidity in the reed.

An embodiment relates to a device comprising a suction channel, a suction connector, a suction tubing, a source of suction force, a processor, and a memory. The device is operable to remove moisture from a reed.

In an embodiment, the device comprises a humidity sensor to measure a value of humidity of the reed.

In an embodiment, the processor is operable to turn on the source of suction force.

In an embodiment, the processor is operable to turn off the source of suction force when a pre-set value of humidity is reached.

In an embodiment, the processor is operable to receive a value of humidity and compare the value of humidity with a pre-set range of humidity. The device maintains the pre-set range of humidity in the reed.

An embodiment relates to a method comprising receiving a first value of humidity of a reed, turning on a device and a source of suction, receiving a second value of humidity of the reed, turning off the source of suction when the reed reaches a pre-set range of humidity. The method is for removing moisture from the reed.

In an embodiment, the first value of humidity is greater than the pre-set range of humidity.

An embodiment relates to an apparatus comprising a spray, an actuator, a water compartment, a memory, and a processor. The processor is communicatively coupled to the memory and wherein the data processor is operable to receive an instruction to actuate the actuator, and activate the spray, and wherein the apparatus is operable to control humidity of a reed stored inside a storage.

In an embodiment, the instruction received is at least one of a time input, a time interval, and a quantity of water to be sprayed.

In an embodiment, the spray comprises at least one of a jet spray, a mist spray, a dripper, and a humidifier.

In an embodiment, the apparatus comprises a humidity sensor to measure the value of humidity of the reed.

In an embodiment, the processor is configured to receive feedback of the humidity inside the apparatus through the humidity sensor.

In an embodiment, the processor is operable to receive the value of humidity, and to compare the value of humidity with a pre-set range of humidity. The apparatus the pre-set range of humidity values in the reed.

In an embodiment, the processor is operable to generate an alert if the value of humidity is lower than the pre-set range of humidity in the reed.

In an embodiment, the apparatus is overridden remotely to turn on the actuator.

In an embodiment, the apparatus further comprises a first sensor to detect at least one of a fungus and moss growth on the reed.

In an embodiment, the apparatus further comprises a second sensor to detect at least one of a crack and a physical damage of the reed.

In an embodiment, the pre-set value of the humidity is set through a user interface.

In an embodiment, the user interface comprises at least one of an interactive display, a mobile device, and a handheld remote device.

In an embodiment, the control of the apparatus is through an application programming interface.

An embodiment relates to a system comprising a memory, and a processor. The processor is communicatively coupled to the memory and wherein the data processor is operable to receive an instruction, actuate an actuator; and activate a spray. The apparatus is operable to control humidity of a reed stored inside a storage.

In an embodiment of the system, the instruction received is at least one of a time input, a time interval, and a quantity of water to be sprayed.

In an embodiment of the system, the spray comprises at least one of a jet spray, a mist spray, a dripper, and a humidifier.

In an embodiment of the system, the system comprises a humidity sensor to measure a value of humidity of the reed.

In an embodiment of the system, the processor is operable to receive the value of humidity, compare the value of humidity with a pre-set range of humidity; and wherein the system maintains the pre-set range of humidity in the reed.

In an embodiment of the system, the processor is operable to generate an alert if the value of humidity is lower than the pre-set range of humidity in the reed.

In an embodiment of the system, the system is overridden remotely to turn on the actuator.

In an embodiment of the system, the system further comprises a first sensor to detect at least one of a fungus and moss growth on the reed.

In an embodiment of the system, the system further comprises a second sensor to detect at least one of a crack and a physical damage of the reed.

An embodiment relates to a method comprising receiving a first value of humidity of a reed, turning on an actuator, receiving a second value of humidity of the reed, turning off the actuator when the reed reaches a pre-set range of humidity. The method is for controlling moisture of the reed.

In an embodiment of the method, the first value of humidity is greater than the pre-set range of humidity.

### BRIEF DESCRIPTION OF THE FIGURES

FIG. 1 shows a perspective view of a device to remove moisture in one embodiment.

FIG. 2 shows a perspective view of the device to remove moisture with a processor in one embodiment.

FIG. 3 shows a perspective view of the device to remove moisture remotely controlled in one embodiment.

FIG. 4A shows a perspective view of another embodiment of the device to remove moisture.

FIG. 4B shows a perspective view of another embodiment of the device to remove moisture.

FIG. 4C shows a perspective view of another embodiment of the device to remove moisture.

FIG. 5 shows a perspective view of another embodiment of the device to remove moisture with the processor.

FIG. 6 shows a perspective view of another embodiment of the device to remove moisture remotely controlled.

FIG. 7 shows a method to remove moisture from a woodwind instrument according to one embodiment.

FIG. 8 shows an embodiment of the device to remove moisture.

FIG. 9 shows a mouthpiece with reed that is fitted with a sensor according to an embodiment.

FIG. 10 shows a perspective view of an apparatus to control moisture with a processor in one embodiment.

FIG. 11 shows a block diagram of the system to control moisture in one embodiment.

FIG. 12 shows a method to control moisture in one embodiment.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

Although the herein detailed description contains many specifics for the purpose of illustration, a person of ordinary skill in the art will appreciate that many variations and alterations to the details are included herein.

Accordingly, the embodiments herein are without any loss of generality to, and without imposing limitations upon, any claims set forth. The terminology used herein is for the purpose of describing embodiments only and is not limiting. Unless defined otherwise, all technical and scientific terms used herein have the same meaning as commonly understood by one with ordinary skill in the art to which this disclosure belongs.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The term “comprising,” which is synonymous with “including,” “containing,” or “characterized by” here as used herein refers to being inclusive or open-ended, and does not exclude additional, unrecited elements or method steps, unless the context clearly requires otherwise.

The terms “couple” “coupled,” “couples,” “coupling,” and the like should be broadly understood and refer to as connecting two or more elements mechanically, electrically, and/or otherwise. Two or more electrical elements may be electrically coupled together, but not be mechanically or otherwise coupled together. Coupling may be for any length of time, e.g., permanent, or semi-permanent or only for an instant. “Electrical coupling” includes electrical coupling of all types. The absence of the word “removably,” “removable,” and the like near the word “coupled,” and the like does not mean that the coupling, etc. in question is or is not removable.

No element act, or instruction used herein is critical or essential unless explicitly described as such. Furthermore, the term “set” includes items (e.g., related items, unrelated items, a combination of related items and unrelated items, etc.) and may be interchangeable with “one or more.” Where only one item is intended, the term “one” or similar language is used. Also, the terms “has,” “have,” “having,” or the like are open-ended terms. Further, the phrase “based on” means “based, at least in part, on” unless explicitly stated otherwise.

As used herein, the terms “system,” “device,” “unit,” and/or “module” refer to a different component, component portion, or component of the various levels of the order. However, other expressions that achieve the same purpose may replace the terms.

As used herein, the terms “left,” “right,” “front,” “back,” “top,” “bottom,” “over,” “under” and the like in the description and in the claims, if any, are for descriptive purposes and not necessarily for describing permanent relative positions. The terms so used are interchangeable under appropriate circumstances such that the embodiments of the apparatus, methods, and/or articles of manufacture described herein are, for example, capable of operation in other orientations than those illustrated or otherwise described herein.

As used herein, the term “or” means an inclusive “or” rather than an exclusive “or.” That is, unless specified otherwise, or clear from context, “X employs A or B” means any of the natural inclusive permutations. That is, if X employs A; X employs B; or X employs both A and B, then “X employs A or B” is satisfied under any of the foregoing instances.

As defined herein, two or more elements or modules are “integral” or “integrated” if they operate functionally together. As defined herein, two or more elements are “non-integral” if each element can operate functionally independently.

Other specific forms may embody the present invention without departing from its spirit or characteristics. The described embodiments are in all respects illustrative and not restrictive. Therefore, the appended claims rather than the description herein indicate the scope of the invention. All variations which come within the meaning and range of equivalency of the claims are within their scope.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As used herein, the term “real-time” refers to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The term “network” as used herein refers to a set of computers sharing resources located on or provided by network nodes. The computers use common communication protocols over digital interconnections to communicate with each other. These interconnections are made up of telecommunication network technologies, based on physically wired, optical, and wireless radio-frequency methods that may be arranged in a variety of network topologies. The nodes of a computer network may include personal computers, servers, networking hardware, or other specialized or general-purpose hosts. The network may include a cloud network.

Implementations may be realized in a computing system that includes a back-end component, e.g., as a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components. The components of the system may be interconnected by any appropriate form or medium of digital data communication, e.g., a communication network. Examples of communication networks include a local area network (LAN) and a wide area network (WAN), e.g., the Intranet and Internet.

The computing system may include clients and servers. A client and server are generally remote from each other and typically interact through a communication network. The relationship of client and server arises by virtue of computer programs running on the respective computers and having a client-server relationship to each other.

Embodiments of the present invention may comprise or utilize a special purpose or general purpose computer including computer hardware. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly distinct kinds of computer-readable media: physical computer-readable storage media and transmission computer-readable media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described. Rather, the described features and acts are disclosed as example forms of implementing the claims.

Processors suitable for the execution of a computer program include, by way of example, both general and special purpose microprocessors, and any one or more processors of any appropriate kind of digital computer. Generally, a processor will receive instructions and data from a read-only memory or a random-access memory or both. Elements of a computer can include a processor for performing instructions and one or more memory devices for storing instructions and data. Generally, a computer will also include, or be operatively coupled to receive data, transfer data or both, to/from one or more mass storage devices for storing data e.g., magnetic disks, magneto optical disks, optical disks, or solid-state disks. However, a computer need not have such mass storage devices. Moreover, a computer may be embedded in another device, e.g., a mobile telephone, a personal digital assistant (PDA), a mobile audio player, a Global Positioning System (GPS) receiver, etc. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disc Read-Only Memory (CD ROM) disks, Digital Versatile Disk-Read-Only Memory (DVD-ROM) disks) and solid-state disks. The processor and the memory may be supplemented by, or incorporated in, special purpose logic circuitry.

To provide for interaction with a user, implementations may be realized on a computer having a display device (e.g., a cathode ray tube (CRT) or liquid crystal display (LCD) monitor) for displaying information to the user and a keyboard and a pointing device, e.g., a mouse or a trackball, by which the user may provide input to the computer. Other kinds of devices may be used to provide for interaction with a user as well; for example, feedback provided to the user may be any appropriate form of sensory feedback, e.g., visual feedback, auditory feedback, or tactile feedback; and input from the user may be received in any appropriate form, including acoustic, speech, or tactile input.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, medical smart storage are described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

Example embodiments, as described below, may be used to provide a system a. It will be appreciated that the various embodiments discussed herein need not necessarily belong to the same group of embodiments and may be grouped into various other embodiments not explicitly disclosed herein. In the following description, for purposes of explanation, numerous specific details are set forth in order to provide a thorough understanding of the various embodiments.

Implementations and all of the functional operations described in this specification may be realized in digital electronic circuitry, or in computer software, firmware, or hardware.

A software program (also known as a program, software, executable code, or instructions) may be written in any appropriate form of programming language, including compiled or interpreted languages, and it may be deployed in any appropriate form, including as a standalone program or as a module, component, subroutine, or other unit suitable for use in a computing environment.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may generally be integrated together in a single software product or packaged into multiple software products.

Even though particular combinations of features are recited in the claims and/or disclosed in the specification, these combinations are not intended to limit the disclosure of possible implementations. Other implementations are within the scope of the following claims. For example, the actions recited in the claims may be performed in a different order and still achieve desirable results. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

Further, the methods may be practiced by a computer system including one or more processors and computer-readable media such as computer memory. In particular, the computer memory may store computer-executable instructions that when executed by one or more processors cause various functions to be performed such as the acts recited in the embodiments.

Digital electronic circuitry, or in computer software, firmware, or hardware, including the structures disclosed in this specification and their structural equivalents, or in combinations of one or more of them may realize the implementations and all of the functional operations described in this specification. Implementations may be as one or more computer program products i.e., one or more modules of computer program instructions encoded on a computer-readable medium for execution by, or to control the operation of, data processing apparatus. The computer-readable medium may be a machine-readable storage device, a machine-readable storage substrate, a memory device, a composition of matter affecting a machine-readable propagated signal, or a combination of one or more of them. The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that encodes information for transmission to a suitable receiver apparatus.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a standalone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as, special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in a non-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specification and drawings are illustrative rather than restrictive.

Physical computer-readable storage media includes RAM, ROM, EEPROM, CD-ROM or other optical disk storage (such as CDs, DVDs, etc.), magnetic disk storage or other magnetic storage devices, solid-state disks or any other medium. They store desired program code in the form of computer-executable instructions or data structures which can be accessed by a general purpose or special purpose computer.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer access transmission media that can include a network and/or data links which carry desired program code in the form of computer-executable instructions or data structures. The scope of computer-readable media includes combinations of the above, that enable the transport of electronic data between computer systems and/or modules and/or other electronic devices.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Module (NIC), and then eventually transferred to computer system RAM and/or to less volatile computer-readable physical storage media at a computer system. Thus, computer system components that also (or even primarily) utilize transmission media may include computer-readable physical storage media.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the invention may be practiced in network computing environments with many types of computer system configurations including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the invention. In a distributed system environment, program modules may be located in both local and remote memory storage devices.

The embodiments described herein can be directed to one or more of a system, a method, an apparatus, and/or a computer program product at any possible technical detail level of integration. The computer program product can include a computer readable storage medium (or media) having computer readable program instructions thereon for causing a processor to carry out aspects of the one or more embodiments described herein. The computer readable storage medium can be a tangible device that can retain and store instructions for use by an instruction execution device. For example, the computer readable storage medium can be, but is not limited to, an electronic storage device, a magnetic storage device, an optical storage device, an electromagnetic storage device, a superconducting storage device, and/or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon and/or any suitable combination of the foregoing. A computer readable storage medium, as used herein, does not construe transitory signals per se, such as radio waves and/or other freely propagating electromagnetic waves, electromagnetic waves propagating through a waveguide and/or other transmission media (e.g., light pulses passing through a fiber-optic cable), and/or electrical signals transmitted through a wire.

Computer readable program instructions described herein are downloadable to respective computing/processing devices from a computer readable storage medium and/or to an external computer or external storage device via a network, for example, the Internet, a local area network, a wide area network and/or a wireless network. The network can comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the one or more embodiments described herein can be assembler instructions, instruction-set-architecture (ISA) instructions, machine instructions, machine dependent instructions, microcode, firmware instructions, state-setting data, configuration data for integrated circuitry, and/or source code and/or object code written in any combination of one or more programming languages, including an object oriented programming language such as Smalltalk, C++ or the like, and/or procedural programming languages, such as the “C” programming language and/or similar programming languages. The computer readable program instructions can execute entirely on a computer, partly on a computer, as a stand-alone software package, partly on a computer and/or partly on a remote computer or entirely on the remote computer and/or server. In the latter scenario, the remote computer can be connected to a computer through any type of network, including a local area network (LAN) and/or a wide area network (WAN), and/or the connection can be made to an external computer (for example, through the Internet using an Internet Service Provider). In one or more embodiments, electronic circuitry including, for example, programmable logic circuitry, field-programmable gate arrays (FPGA), and/or programmable logic arrays (PLA) can execute the computer readable program instructions by utilizing state information of the computer readable program instructions to personalize the electronic circuitry, in order to perform aspects of the one or more embodiments described herein.

Aspects of the one or more embodiments described herein are described with reference to flowchart illustrations and/or block diagrams of methods, apparatus (systems), and computer program products according to one or more embodiments described herein. Each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer and/or other programmable data processing apparatus to produce a machine, such that the instructions, which execute via the processor of the computer or other programmable data processing apparatus, can create means for implementing the functions/acts specified in the flowchart and/or block diagram block or blocks. These computer readable program instructions can also be stored in a computer readable storage medium that can direct a computer, a programmable data processing apparatus and/or other devices to function in a particular manner, such that the computer readable storage medium having instructions stored therein can comprise an article of manufacture including instructions which can implement aspects of the function/act specified in the flowchart and/or block diagram block or blocks. The computer readable program instructions can also be loaded onto a computer, other programmable data processing apparatus and/or other device to cause a series of operational acts to be performed on the computer, other programmable apparatus and/or other device to produce a computer implemented process, such that the instructions which execute on the computer, other programmable apparatus and/or other device implement the functions/acts specified in the flowchart and/or block diagram block or blocks.

The flowcharts and block diagrams in the figures illustrate the architecture, functionality and/or operation of possible implementations of systems, computer-implementable methods and/or computer program products according to one or more embodiments described herein. In this regard, each block in the flowchart or block diagrams can represent a module, segment and/or portion of instructions, which comprises one or more executable instructions for implementing the specified logical function(s). In one or more alternative implementations, the functions noted in the blocks can occur out of the order noted in the Figures. For example, two blocks shown in succession can be executed substantially concurrently, and/or the blocks can sometimes be executed in the reverse order, depending upon the functionality involved. It will also be noted that each block of the block diagrams and/or flowchart illustration, and/or combinations of blocks in the block diagrams and/or flowchart illustration, can be implemented by special purpose hardware-based systems that can perform the specified functions and/or acts and/or carry out one or more combinations of special purpose hardware and/or computer instructions.

While the subject matter described herein is in the general context of computer-executable instructions of a computer program product that runs on a computer and/or computers, those skilled in the art will recognize that the one or more embodiments herein also can be implemented in combination with one or more other program modules. Program modules include routines, programs, components, data structures, and/or the like that perform particular tasks and/or implement particular abstract data types. Moreover, other computer system configurations, including single-processor and/or multiprocessor computer systems, mini-computing devices, mainframe computers, as well as computers, hand-held computing devices (e.g., PDA, phone), microprocessor-based or programmable consumer and/or industrial electronics and/or the like can practice the herein described computer-implemented methods. Distributed computing environments, in which remote processing devices linked through a communications network perform tasks, can also practice the illustrated aspects. However, stand-alone computers can practice one or more, if not all aspects of the one or more embodiments described herein. In a distributed computing environment, program modules can be located in both local and remote memory storage devices.

As used in this application, the terms “component,” “system,” “platform,” “interface,” and/or the like, can refer to and/or can include a computer-related entity or an entity related to an operational machine with one or more specific functionalities. The entities described herein can be either hardware, a combination of hardware and software, software, or software in execution. For example, a component can be, but is not limited to being, a process running on a processor, a processor, an object, an executable, a thread of execution, a program and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system and/or across a network such as the Internet with other systems via the signal). As another example, a component can be an apparatus with specific functionality provided by mechanical parts operated by electric or electronic circuitry, which is operated by a software and/or firmware application executed by a processor. In such a case, the processor can be internal and/or external to the apparatus and can execute at least a part of the software and/or firmware application. As yet another example, a component can be an apparatus that provides specific functionality through electronic components without mechanical parts, where the electronic components can include a processor and/or other means to execute software and/or firmware that confers at least in part the functionality of the electronic components. In an aspect, a component can emulate an electronic component via a virtual machine, e.g., within a cloud computing system.

As it is employed in the subject specification, the term “processor” can refer to any computing processing unit and/or device comprising, but not limited to, single-core processors; single-processors with software multithread execution capability; multi-core processors; multi-core processors with software multithread execution capability; multi-core processors with hardware multithread technology; parallel platforms; and/or parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, and/or any combination thereof designed to perform the functions described herein. Further, processors can exploit nano-scale architectures such as, but not limited to, molecular based transistors, switches and/or gates, in order to optimize space usage and/or to enhance performance of related equipment. A combination of computing processing units can implement a processor.

Herein, terms such as “store,” “storage,” “data store,” data storage,” “database,” and any other information storage component relevant to operation and functionality of a component refer to “memory components,” entities embodied in a “memory,” or components comprising a memory. Memory and/or memory components described herein can be either volatile memory or nonvolatile memory or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, and/or nonvolatile random access memory (RAM) (e.g., ferroelectric RAM (FeRAM). Volatile memory can include RAM, which can function as external cache memory, for example. By way of illustration and not limitation, RAM can be available in many forms such as synchronous RAM (SRAM), dynamic RAM (DRAM), synchronous DRAM (SDRAM), double data rate SDRAM (DDR SDRAM), enhanced SDRAM (ESDRAM), Synch link DRAM (SLDRAM), direct Rambus RAM (DRRAM), direct Rambus dynamic RAM (DRDRAM) and/or Rambus dynamic RAM (RDRAM). Additionally, the described memory components of systems and/or computer-implemented methods herein include, without being limited to including, these and/or any other suitable types of memory.

The embodiments described herein include mere examples of systems and computer-implemented methods. It is, of course, not possible to describe every conceivable combination of components and/or computer-implemented methods for purposes of describing the one or more embodiments, but one of ordinary skill in the art can recognize that many further combinations and/or permutations of the one or more embodiments are possible. Furthermore, to the extent that the terms “includes,” “has,” “possesses,” and the like are used in the detailed description, claims, appendices and/or drawings such terms are intended to be inclusive in a manner similar to the term “comprising” as “comprising” is interpreted when employed as a transitional word in a claim.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

The following terms and phrases, unless otherwise indicated, shall be understood to have the following meanings.

The term “communication module” is a module that facilitates communication, that is, it enables transmission and receiving of data from the input and output interfaces to the processor. It also enables communication between the peripheral devices connected with the processor like display, camera, remote servers, and databases. A communication module may be a wired connection between the components or a wireless communication module.

The term “sensor” as used herein refers to a device that detects or measures a physical property and enables the recording, presentation or response to such detection or measurement using processor and optionally memory. A sensor and processor can take one form of information and convert such information into another form, typically having more usefulness than the original form.

As used herein, the term “component” is intended to be broadly construed as hardware, firmware, and/or a combination of hardware and software.

As used herein, the term “API” stands for Application Programming Interface. It is an interface that defines interactions between multiple software applications or mixed hardware-software intermediaries. It defines the kinds of calls or requests that can be made, how to make them, the data formats that should be used, the conventions to follow, etc. It can also provide extension mechanisms so that users can extend existing functionality in several ways and to varying degrees. An API can be entirely custom, specific to a component, or designed based on an industry-standard to ensure interoperability. Through information hiding, APIs enable modularity, allowing users to use the interface independently of the implementation. Web APIs is now the most common meaning of the term API. There are also APIs for programming languages, software libraries, computer operating systems, and computer hardware.

The term “communicatively coupled” as used herein refers to devices connected in a way that permits communication.

The term “controller” as used herein refers to the component of a system that functions as the system controller. A controller typically sends program messages to and receives response messages from devices. A functional unit in a computer system that controls one or more units of the peripheral equipment. Synonym: peripheral control unit. See also: input-output controller; dual channel controller. In robotics, a controller takes as input desired and measured position, velocity or other pertinent variables and whose output is a drive signal to a controlling motor or activator. A device through which one can introduce commands to a control system.

The term “fan” as used herein refers to a fan that sucks or draws the air toward it through airways or air pipes. The term generally used is exhaust fan or a suction fan.

The term “desiccant” as used herein refers to a substance or chemical that absorbs or attracts moisture causing a state of dryness (desiccation) in its vicinity. This chemical is used to attract and remove moisture from air or gas and keep the products dry.

The term “handheld device” as used herein refers to any portable device that can be carried and held in one's palm.

The term “moisture” as used herein refers to water or other liquid diffused in a small quantity as vapor, within a solid, or condensed on a surface.

The term “musical instrument” as used herein refers to a device, object, contrivance, or machine used to produce musical notes or sounds. Here musical instrument refer to woodwind instruments or instruments that need to be played by mouth.

The term “woodwind instrument” as used herein refers to any of a group of wind musical instruments, composed of the flutes and reed pipes i.e., clarinet, oboe, bassoon, and saxophone. Both groups were traditionally made of wood, but now they may also be constructed of metal.

The term “suction channel” as used herein refers to passage within a device through which fluids may be injected or removed. Here the suction channel is used to draw moisture away from the woodwind instrument to make the reeds dry.

The term “proximal end” as used herein refers to towards or nearest to the point of attachment or the point of origin of a part. Example, the proximal end of the suction channel attaches to the woodwind instrument. Here the proximal end of the suction channel is the end that attaches to the mouthpiece of the woodwind instrument.

The term “recess” or “suction tubing recess” as used herein refers to a receding or hollow place, as in a surface to fix something on to the recess.

The term “mouthpiece” as used herein refers to a part of a musical instrument, telephone, etc. designed to be put in or against the mouth.

The term “reed” as used herein refers to a thin strip of material that vibrates to produce a sound on a musical instrument. Most woodwind instrument reeds may be made from Arundo donax (Giant cane) or synthetic material. Tuned reeds as in harmonicas and accordions, are made of metal or synthetics. Musical instruments are classified according to the type and number of reeds.

The term “flexible material” as used herein refers to material that is characterized by the ability to bend or compress easily without cracking under normal conditions.

The term “compartment” as used herein refers to a separate section or part of a structure or container. Here it refers to a separate part of the suction channel separated by a thin breathable layer.

The term “distal end” as used herein refers to the part located away from a specific area, most often away from the center of any body. Here the distal end of the suction channel is the end that is attached to the compartment comprising the desiccant.

The term “switch” as used herein refers to a device used to make or break a connection in a circuit so you can turn power on and off to something. An example of a switch is the switch on the wall used to turn a light on and off.

The term “suction” or “suction force” as used herein refers to a force created when there's a difference in air pressure. Removing air from a space results in a pressure differential. Suction pressure is therefore limited by external air pressure.

The term “fan blade” as used herein refers to specially shaped rotating parts (blades) of a mechanical fan which move, distribute, or provide suction of air.

The term “suction connector” as used herein refers to a connector that joins two pieces of equipment or piping together. Here the suction connector connects the source of suction and the suction channel together.

The term “suction tubing” as used herein refers to a device which provides suction by being attached to a suction machine. Suction can be used to clear liquids and slurries away from an area of concern. Here the suction tubing is attached to the suction channel to provide efficient suction.

The term “source of a suction force” as used herein refers to a device that draws air or other gas molecules from a sealed volume in order to leave behind a partial vacuum and provide suction. The job of a source of suction force is to generate a relative vacuum within a capacity.

The term “humidity control pouch” as used herein refers to a pouch containing natural absorbents to absorb moisture. An example of a humidity control pouch is a Boveda® pack. The saturation level of certain salts in water naturally regulates the humidity in the immediate environment to a pre-specified relative humidity level. When placed in a container or package, the Boveda® pack either emits or draws in moisture from the surrounding air, keeping the optimal humidity level, protects woodwind instruments from fluctuating humidity and damage.

The term “music stand” as used herein refers to a pedestal or elevated rack designed to hold a paper score or sheets of music in position for reading. A music stand may also refer to a pedestal to hold the musical instrument. Most music stands for orchestral, chamber music or solo orchestra-family instruments violin, oboe, trumpet, etc. can be raised or lowered to accommodate seated or standing performers, or performers of different heights.

The term “humidity sensor” as used herein refers to an electronic device that measures the humidity in its environment and converts its findings into a corresponding electrical signal.

An embodiment relates to a device comprising a suction channel, a fan, and a desiccant. The device is a handheld device operable to remove moisture from a musical instrument. The fan is connected to a proximal end of the suction channel. The device further comprises a recess. The device is attachable to a woodwind instrument through the recess to remove moisture from a reed of the woodwind instrument. The recess is made of flexible material that fixes the device on the mouthpiece of varying size.

In an embodiment, the device is attachable to a mouthpiece of the woodwind instrument.

“A woodwind reed storage and maintenance device conveniently fits directly onto the reed bearing end of a musical instrument. The storage and maintenance device includes a reed storage space, humidity control device space, and a humidity gauge. The storage and maintenance device is used to maintain a reed while still located on the musical instrument at a specifically chosen constant humidity level for optimal performing condition and maximum reed life. The storage and maintenance device protects the reed from physical damage and from damage due to changing humidity levels in the ambient surroundings. The storage and maintenance device accurately measures and displays temperature and humidity information corresponding to the reed storage space inside the device.” [Source: Humidity controlled musical instrument reed cap; published as U.S. Ser. No. 10/832,641B2 on Nov. 10, 2020]

“A storage and maintenance apparatus for a musical instrument such as a woodwind or bagpipes comprises a housing 102 for surrounding a reed, the housing including a humidity control device such as silica gel, a sponge, or a two-way humidity control pouch (Boveda®) and an electronic digital hygrometer and/or thermometer 100 preferably with an LCD display. Preferably the housing has a body 102 and cap 100 at one end and an opening at the other end, the cap containing the hygrometer and/or thermometer. The body being cylindrical in shape with an internal humidity control device holder 108 for creating a reed storage area 104 and separating the humidity control device from contacting the reed (not shown). The body is preferably made from silicone material and sized to create a snug fit with a chanter or musical instrument when fitted. A lock screw 106 may be provided for securing the apparatus to the musical instrument.” [Source: Musical instrument read storage and maintenance device with humidity control and digital hygrometer and thermometer; published as GB2565207A on Nov. 27, 2019]

“Methods of improving reed performance are provided comprising the steps of treating one or more reeds with a solution and cycling the pressure over the solution from atmospheric pressure to reduced pressure to atmospheric pressure.” [Source: Musical reed treatments; published as WO2020263918A1 on Dec. 30, 2020]

“A cap for maintaining the reed of musical instruments moist when not in use comprising a cone shaped housing having an open end. An elongated slot is formed in the side wall of the housing. An offset compartment adjacent to the closed end of the housing is provided for a flexible moisture pad. Means is provided for frictionally holding the mouthpiece in the cap with the reed confronting and engaging the moisture pad to wet the reed when the instrument is not in use.” [Source: Moisture cap assembly for reed instruments; published as US20070221039A1 on Sep. 27, 2007]

There is a need for a device that removes moisture from the woodwind instruments that solve the problem of the prior art. There is a need for a device that removes the excess moisture from the reeds and maintains an optimum moisture level to prevent the reed from any damage due to the moisture.

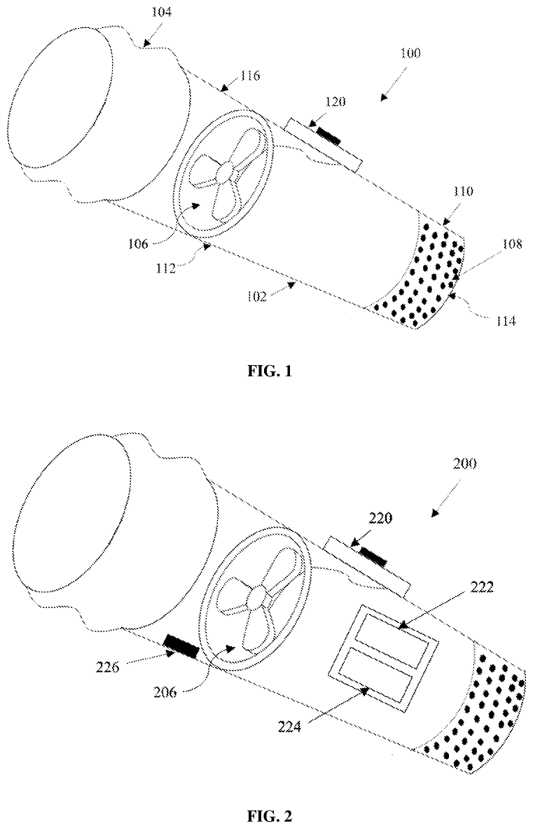

FIG. 1 shows a perspective view of a device to remove moisture in one embodiment. The device 100 comprises a suction channel 102, a recess 104, a fan 106 and a desiccant 108. The suction channel 102 is inside the device 100. The suction channel 102 comprises a proximal end 112 and a distal end 114. The fan 106 is connected to the proximal end 114 of the suction channel 102. The end of the suction channel that is in proximity with the reed is referred to as a proximal end 112 of the suction channel 102. The end of the suction channel that is at another end or away from the reed is referred as a distal end. The device 100 further comprises a recess 104. The recess 104 is attached to the device through a tubing 116. The tubing 116 is connected around the fan 106.

In an embodiment, the device 100 is attachable to a woodwind instrument through the recess 104 to remove moisture from a reed of the woodwind instrument. The recess 116 may be made of flexible material that fixes the device on the mouthpiece of the woodwind instrument of varying size. The flexible material comprises at least one of a natural rubber, elastane or Lycra®, butyl rubber (GDP), fluor elastomer, elastomers, ethylene-propylene rubber (EPR), resin, styrene-butadiene rubber (SBR), chloroprene, elastin, rubber epichlorohydrin, nylon, nylon13-terpene, isoprene rubber, polybutadiene, nitrile rubber, stretchable vinyl, thermoplastic elastomer, silicone rubber, ethylene-propylene-diene rubber (EPDM), ethyl vinyl acetate (EVA rubber or foam), halogenated butyl rubbers (CIIR, BIIR), neoprene.

In an embodiment, a compartment at a distal end of the suction channel comprises the desiccant. The fan removes the moisture by sucking the moisture into the compartment comprising the desiccant. The fan is operated using a switch. The fan is a suction fan. A fan blade is designed at an angle to pull out the moisture from the woodwind instrument.

In an embodiment the device 100 comprises a compartment 110 at a distal end of the suction channel 102. The compartment 110 contains the desiccant 108. The compartment 110 is separated from the suction channel 102 by a thin breathable layer that allows the desiccant to absorb the moisture. The thin breathable layer may be made of hydrophobic material like plastic net. The thin breathable layer may be a net like structure that allows moisture to pass in and does not allow the desiccant to fall out in the suction channel 102.

In an embodiment, the fan 106 removes the moisture by suction of the moisture into the compartment 110 comprising the desiccant 108. The fan 106 is operated using a switch 120. The fan 106 is a suction fan. The device 100 is a handheld device of any shape. The device fits on the mouthpiece of the woodwind instrument through the recess 104. The device fixes on the mouthpiece as a cap. Once the device is fixed, the fan 106 is operated through a switch 120. The fan pulls out the moisture from the reed of the wood wind instrument. The fan passes the moisture by suction to the compartment 110 containing the desiccant. The desiccant absorbs the excess moisture to keep the reed dry and avoid damage to the reed. The desiccant may comprise a drying-agent such as silica gel, or a humidity control pouch. A humidity control pouch protects woodwind instruments from fluctuating humidity and damage.

In an embodiment, a fan blade designed at an angle to pull out the moisture from the woodwind instrument. The fan blade is designed such that it improves the aerodynamic performance of the axial suction fan; a new type of blade is designed using the equal-variable circulation method. The equal circulation design method improves the aerodynamic performance of the blade roots, while the variable circulation design method enhances the aerodynamic performance of the blade tips. By adopting the equal-variable circulation design method, the total pressure of the fan is increased, while the efficiency remains unchanged. Forward-swept blades with an equal-variable circulation design also improve performance over the conventional blades by changing the center-of-gravity stacking line. At low flow rates, the efficiency of the fan can be increased, and the working range of the flow is expanded.

In an embodiment, the device comprises a humidity sensor to measure a value of humidity of the reed. The device comprises a processor and a memory. The processor is operable to operate the device. The processor is operable to receive the value of humidity and compare the value of humidity with a pre-set range of humidity. The device maintains the pre-set range of humidity in the reed. The processor is operable to turn on the source of suction force. The processor is operable to turn off the source of suction force when a pre-set value of humidity is reached. The processor is operable to receive a value of humidity, compare the value of humidity with a pre-set range of humidity, and the device maintains the pre-set range of humidity in the reed and avoids damage to the reed.

FIG. 2 shows a perspective view of the device to remove moisture with a processor in one embodiment. The device 200 further comprises a processor 222, a memory 224 and a humidity sensor 226. The processor 222 is configured to control the moisture of the reed. The processor 222 receives a feedback from the humidity sensor 226, that is a value of the moisture in the reed. The processor 222 receives a value of humidity of the reed. The processor 222 further compares the value of humidity with a pre-set range of humidity for the reed. If the value of humidity is more than the pre-set value, the processor 222 turns on the fan 206 with the help of the switch 220. Again, the processor receives the value of humidity and compares the value of humidity with the pre-set range of humidity. In the case if the value of humidity is within the pre-set range of humidity, the processor 222 is operable to switch off the fan 206. Thus, the device maintains the pre-set range of humidity in the reed to keep the reed not in dry condition and avoids damage to the reed.

In an embodiment, the processor 222 is only operable to turn off the fan upon receiving a value of humidity from the humidity sensor. A user may turn on the device with a switch. The processor 222 receives the value of humidity of the reed. The processor 222 further compares the value of humidity with a pre-set range of humidity for the reed. Now if the value of humidity is within the pre-set range of humidity, the processor 222 is operable to switch off the fan 206. The pre-set range of humidity is the optimum humidity range for a reed to function without any damage. The pre-set range is set into the device at the time of manufacturing the device.

FIG. 3 shows a perspective view of the device to remove moisture remotely controlled in one embodiment. The device 300 further comprises a processor 322, a memory 324, a humidity sensor 326, communication module 328, and a remote control device 330. The remote control device may be a remote with on off buttons, a mobile operated app, and a computer controlled app. The processor 322 is further operable to receive the value of humidity of the reed. The processor 322 further compares the value of humidity with a pre-set range of humidity for the reed. The processor 322 generates an alert and communicates to the remote control device 330 through the communication module 328 to turn on or turn off the fan based on the value of humidity received. If the value of humidity is more than the pre-set value, the processor 322 generates an alert to turn on the fan 306. In the case if the value of humidity is within the pre-set range of humidity, the processor 322 generates an alert to turn off the fan 306. The user in this way may control multiple devices 300 with the help of remote control device 330. For example, if there are four devices: device A, device B, device C and device D attached to four different woodwind instruments. Suppose the humidity range of device B and device C is high, the processors of device B and device C generates an alert and communicates with a remote control device at the user's end. Therefore, a user may turn on the fan of device B and device C to pull out the moisture from the reed. The fan of device B and device C may be then turned off by the processor when the processor receives the value of humidity, and the value of humidity is in the range of pre-set value of humidity when compared. In another embodiment, the processor may generate an alert on the remote control device through the communication module to turn off device B and device C, when the processor receives the value of humidity, and the value of humidity is in the range of pre-set value of humidity when compared.

An embodiment relates to a device comprising a suction channel, a suction connector, a suction tubing, and a source of a suction force. The device is operable to remove moisture from a reed. The device is an elongated device comprising a recess at a proximal end of the device operable to attach the device on a mouthpiece of a woodwind instrument. The suction channel is inside the device. The suction connector is connected at a distal end of the device. The suction tubing connected between one end of the suction channel and the recess. The source of the suction force is attached to the suction connector in order to provide the suction force.

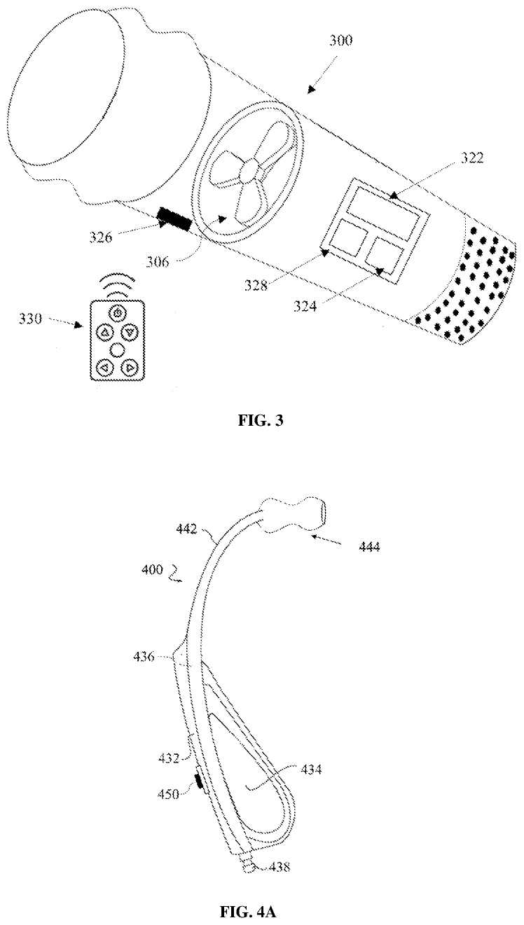

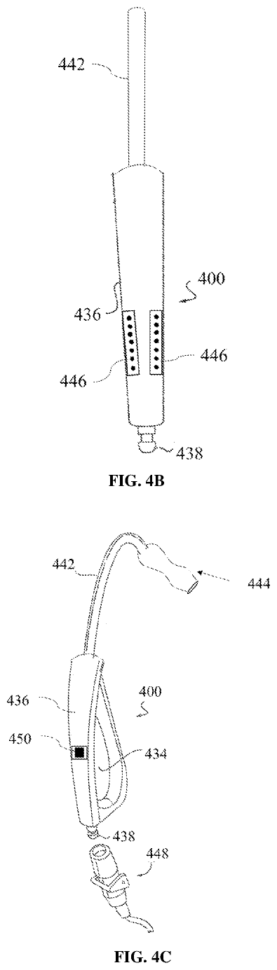

The FIG. 4A-4C shows a perspective view of another embodiment of the device to remove moisture. Referring now to FIGS. 4A-4C, a device for moisture removal 400 may include suctioning component(s) to provide suction through the device 400. The components of the device for moisture removing 400 may be the same or similar in many respects to that of the other device for moisture removing as described herein and vice versa.

Suction may be provided via a built-in suction channel within a handle of the device 400. The device 400 may include a handle 432, which may include a hand hold 434, a suction channel 436 in the handle 432, and a suction connector 438 at the distal end of the handle 432. As illustrated in FIG. 4C, a piece of suction tubing 442 may be connected to one end of the suction channel 436, and a source of suction force 448 may be attached to the suction connector 438, in order to provide suction within the woodwind instrument during pulling out the moisture. The suction channel 436 in the handle 432 may extend from a suction tubing 442 at one end to the suction connector 438 at the other end. The suction tubing 442 is narrower than the suction channel 436 and is in the shape of a pipe. The narrow pipe like structure helps in providing efficient suction from the reed as the surface area of the suction tubing 442 is lesser than the suction channel 436.

FIG. 4B is a frontal view of the device 400. At the junction of the suction tubing 442, the suction tubing recess 444 may be configured to receive a proximal end of a piece of suction tubing 442, preferably with a friction fit. The device 400 is fitted onto the woodwind instrument through the suction tubing recess 444.

In an embodiment, the device 400 is attachable to a woodwind instrument through the recess 444 to remove moisture from a reed of the woodwind instrument. The suction tubing recess 444 is made of flexible material that fixes the device on the mouthpiece of the woodwind instrument of varying size. The flexible material comprises at least one of a Natural rubber, Elastane or Lycra®, Butyl rubber (GDP), Fluor elastomer, Elastomers, Ethylene-propylene rubber (EPR), Resin, Styrene-butadiene rubber (SBR), Chloroprene, Elastin, Rubber epichlorohydrin, Nylon, Nylon13-Terpene, Isoprene rubber, Polybutadiene, Nitrile rubber, Stretchable vinyl, Thermoplastic elastomer, Silicone rubber, Ethylene-propylene-diene rubber (EPDM), Ethyl vinyl acetate (EVA rubber or foam), Halogenated butyl rubbers (CIIR, BIIR), Neoprene.

In an embodiment, the source of the suction force comprises at least one of a suction pump, a vacuum pump, an air pressure pump, and a fan. The suction force is adjusted to dry the reed or the woodwind instrument.

A positive displacement vacuum pump operates by expanding and contracting a sealed chamber where the flow of fluid is controlled by one-way valves. The vacuum generation process starts by expanding a sealed chamber generating a vacuum. This vacuum draws the fluid into the chamber through an intake valve. Upon reaching the maximum expansion, the intake valve closes while the exhaust opens. The fluid is ejected out of the chamber as it compresses or contracts. The cycle repeats several times per second, itself creating a pulsating flow. The vacuum generated in the vacuum pumps used herein is of low pressure such that it does not damage the reed or the parts of the woodwind instrument. The suction force of the vacuum pump may be adjusted according to the requirement of the woodwind instrument.

In an embodiment, the device comprises a desiccant. The desiccant comprises at least one of a silica gel and a humidity control pouch.

In an embodiment, the device is connected to a music stand. The device is operated by a switch.

As shown in FIG. 4B, the suction channel 436 contains the desiccant 446. The desiccant 446 is provided at the sides of the suction channel 436. The desiccant 446 is separated from the suction channel 436 by a thin breathable layer that allows the desiccant to absorb the moisture. The thin breathable layer may be made of hydrophobic material like plastic. The thin breathable layer may be a net like structure that allows moisture to pass in and does not allow the desiccant 446 to fall out in the suction channel 436.

In an embodiment, the device 400 removes the moisture by suctioning the moisture into the suction channel comprising the desiccant 446. The device may be operated using a switch 450. The source of the suction force 448 comprises at least one of a suction pump, a vacuum pump, an air pressure pump, and a fan. The device 400 is a handheld device of any shape. The device may be affixed to a music stand that holds the woodwind instrument. When there is a need for removing the moisture from the woodwind instrument, the device on the music stand is fixed on the mouthpiece of the woodwind instrument through the suction tubing recess 444. Once the device is fixed, the source of suction 448 is turned on through a switch 450. The source of suction 448 pulls out the moisture from the reed of the wood wind instrument. The source of suction 448 passes the moisture by suction to the suction channel 436 containing the desiccant 446. The desiccant 446 absorbs the excess moisture to keep the reed dry and avoid damage of the reed. The desiccant 446 may comprise drying-agents such as silica gel, or a humidity control pouch. A humidity control pouch protects woodwind instruments from fluctuating humidity and damage.

An embodiment relates to a device comprising a suction channel, a suction connector, a suction tubing, a source of suction force; a processor; and a memory. The device is operable to remove moisture from a reed. The device comprises a humidity sensor to measure the value of humidity of the reed. The processor is operable to turn on the source of suction force. The processor is operable to turn off the source of suction force when a pre-set value of humidity is reached. The processor is operable to receive a value of humidity, compare the value of humidity with a pre-set range of humidity, and the device maintains the pre-set range of humidity in the reed.

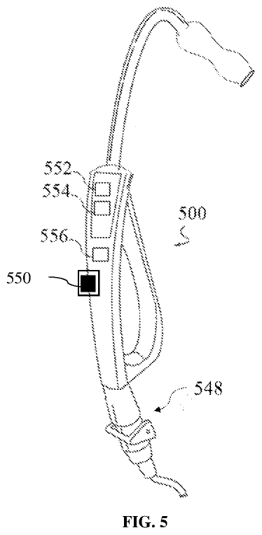

FIG. 5 shows a perspective view of the device to remove moisture with a processor in one embodiment. The device 500 further comprises a processor 552, a memory 554 and a humidity sensor 556. The processor 552 receives a value of humidity through the humidity sensor 556. The processor 552 controls the moisture with feedback received from the humidity sensor 556. The processor 552 receives a value of humidity of the reed of the woodwind instrument, when the device is fixed on the mouthpiece of the woodwind instrument. The processor 552 further compares the value of humidity with a pre-set range of humidity for the reed. If the value of humidity being more than the pre-set range, the processor 552 turns on the source of suction 548 with the help of the switch 550. Again, the processor 552 receives the value of humidity and compares the value of humidity with the pre-set range of humidity. In the case if the value of humidity is within the pre-set range of humidity, the processor 552 is operable to switch off the source of suction 548. Thus, the device maintains the pre-set range of humidity in the reed dry and avoids damage to the reed.

In an embodiment, the processor 552 is only operable to turn off the source of suction 548 upon receiving a value of humidity from the humidity sensor 556. A user may first turn on the device with a switch. The processor 552 is further operable to receive the value of humidity of the reed through the humidity sensor 556. The processor 552 further compares the value of humidity received from the humidity sensor 556 with a pre-set range of humidity for the reed. Now if the value of humidity is within the pre-set range of humidity, the processor 552 is operable to switch off the source of suction 548.

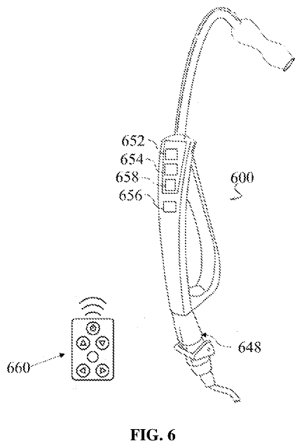

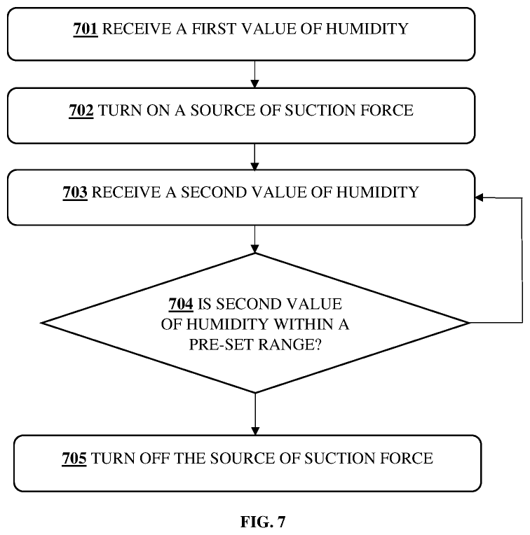

FIG. 6 shows a perspective view of the device to remove moisture remotely controlled in one embodiment. The device 600 further comprises a processor 652, a memory 654, a humidity sensor 656, communication module 658, and a remote control device 660. The remote control device 660 may be a remote with on off buttons, a mobile operated app, and a computer controlled app to remotely control the operation of the device. The processor 652 is further operable to receive the value of humidity of the reed. The processor 652 further compares the value of humidity with a pre-set range of humidity for the reed. The processor 652 generates an alert and communicates to the remote control device 660 through the communication module 658 to turn on or turn off the source of suction based on the value of humidity received. If the value of humidity being more than the pre-set range, the processor 652 generates an alert to turn on the source of suction 648. In the case if the value of humidity is within the pre-set range of humidity, the processor 652 generates an alert to turn off the source of suction 648. The user in this way may control multiple devices 600 with the help of remote control device 660. For example, there are four devices: device A, device B, device C and device D attached to four different woodwind instruments. Suppose the humidity range of device B and device C is high, the processors of device B and device C generates an alert and communicates with a remote control device at the user's end. Therefore, a user may turn on the source of suction 648 of device B and device C to pull out the moisture from the reed. The source of suction of device B and device C may be then turned off by the processor when the processor receives the value of humidity, and the value of humidity is in the range of pre-set value of humidity when compared. In another embodiment, the processor may generate an alert on the remote control device through the communication module to turn off device B and device C, when the processor receives the value of humidity, and the value of humidity is in the range of pre-set value of humidity when compared.

In an embodiment, the device comprises a sensor to detect if the reed of the instrument is cracked or damaged.

An embodiment relates to a method comprising receiving a first value of humidity of a reed, turning on a device and a source of suction, receiving a second value of humidity of the reed, and turning off the source of suction when the reed reaches a pre-set range of humidity. The method is for removing moisture from the reed. The first value of humidity is greater than the pre-set range of humidity.

FIG. 7 shows a method for removing moisture from the reed.

At step 701, the first value of humidity of the reed is received through a humidity sensor. The first value of humidity is then compared with a pre-set range of humidity for the reed.

At step 702, a source of suction is turned on when the first value of humidity is more than the pre-set range of humidity.

At step 703, the second value of humidity of the reed is received through a humidity sensor.

At step 704, the second value of humidity is then compared with a pre-set range of humidity for the reed.

At step 705, a source of suction is turned off when the second value of humidity is within the pre-set range of humidity.

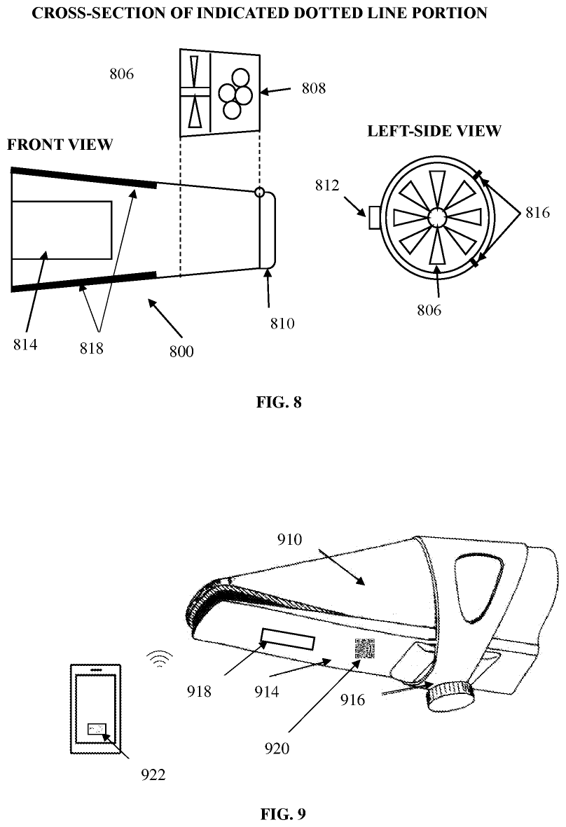

In an embodiment, the mouthpiece of the woodwind instrument is either a plastic or hard rubber piece workpiece where an artist blows through. The sound is produced by a piece of cane which is a reed, a thin strip of cane, it is placed onto the mouthpiece and there is a small slit between the mouthpiece of the woodwind instrument and the reed where the air flows through and that causes the cane to vibrate. The reed held in place with a metal brace called the ligature. The reed vibrates much more easily when it is moist and saturated with moisture. However, excess buildup of moisture during the performance can cause interruptions in the sound and produce a fuzzy or unclear sound with crackling, which is not desirable in a professional setting for performances. In order to prevent excess moisture buildup, a device as shown in FIG. 8, which is a moisture removing device 800 that fits onto the reed, is designed. The device 800 comprises a fan 806, a small fan 806 that would suck the moisture out for a period of time the device is switched on, for e.g., one to five seconds maximum. The moisture once sucked out of the reed goes into a chamber 808 for storage of desiccant in the back of the back of the cap or device comprising a desiccant such as silica gel or some other desiccant to absorb the moisture for later disposal. The chamber 808 comprises a hinged door 810, allowing the user to access the chamber to remove and replace the contents (desiccant) of the chamber. In an embodiment, the chamber is transparent and has an indicator sensor which may provide color indication of when the desiccant is saturated and needs to be replaced. In an embodiment, an alert may be provided. The device comprises a button 812 to activate the fan 806. The button or switch can be activated either manually or using a preset timer. In an embodiment, it may be activated using a remote controller that can control the operation of the switch/button 812. Slot 814 is a section of the cylindrical piece that is cut out so that the ligature, which is attached onto and extends out from the mouthpiece, can be kept unobstructed. The ligature is a small metal/rubber fastener used to the tighten reeds onto the mouthpiece. It is kept on while playing and is removable independent of the mouthpiece itself. The two marks as shown at 816 indicate where the cuts would be made to remove a section of the cylindrical piece to allow the ligature to fit. In an embodiment, the device 800 may optionally comprises a built-in Ultra-Violet (UV) sterilization unit 818. The UV system helps kill the bacteria and fungi on the reed's surface.

FIG. 9 shows a mouthpiece with reed that is fitted with a sensor according to an embodiment. In an embodiment, a small, lightweight sensor 918 attaches to the reed 914 of the mouthpiece 910 itself. In an embodiment, an identifier code 920 is fixed on each reed. The reed is secured with a screw ligature 916 to the mouthpiece 910. The sensor 918 is operable to measure various factors, including one or more moisture levels, temperature, and the reed's vibrations during play. The sensor sends this data to a smartphone app 922 via Bluetooth®. The smartphone app provides real-time feedback on the reed's condition. The app may suggest when the moisture needs to be removed according to an embodiment based on the quality of the sound the reed is making. Further the vibration data is analyzed using various algorithms in the smartphone app to track the need for replacement of the reed based on the damage to the reed. The app further tracks how many hours the reed has been used using the identifier code 920, which may be a Quick Response (QR) code and offers recommendations based on its performance history. The app can also suggest reed rotation schedules to balance wear across multiple reeds. The app may further connect to a database of reed manufacturers, allowing musicians to track the quality and consistency of reeds from different brands. This helps users make informed decisions when purchasing new reeds. In an embodiment, the app collects data on a musician's playing habits and the impact on reeds. It can provide insights into how the player's technique affects reed lifespan and sound quality.

The reeds in all woodwind instruments require moisture to vibrate easily and produce sound. The system prevents too much moisture from collecting on the reed. However, the opposite is also true. Reeds are hardly playable when they are dry, which is often the case when they have been left for long after playing the instrument. This is especially problematic during the winter months when the environment is colder and drier. Currently, musicians prepare their reeds for practice/performance by removing the reed from the case and allowing it to soak in a small amount of water for about 10 minutes. However, this can become tedious, especially when musicians are given a short window of time to get ready to play.

The system comprises a reed case (for reed storage and convenient for carrying) that comprises small jets to spray tiny amounts of water onto the reeds. The case will contain a compartment for water that the user may change and wash. The case further comprises a screen where the musician can program a specific time for the case to spray the reeds or give a frequency to consistently spray the reeds so that they are ready at any time in near future. The system may comprise spray in the form of small jets, inside of the case that would spray mist of water onto the reeds in the case at a pre-set time by the musician. The system comprises a user interface or a screen on the case where the user programs a certain time during the day to do some number of puffs of moisture, the user may select multiple times for one day. In an embodiment, the system comprises a manual option in case of urgency for e.g., the user has a 20-minute window to get ready. Therefore, the system may be operated by a manual switch on the case and reeds are saturated with moisture and ready to play.

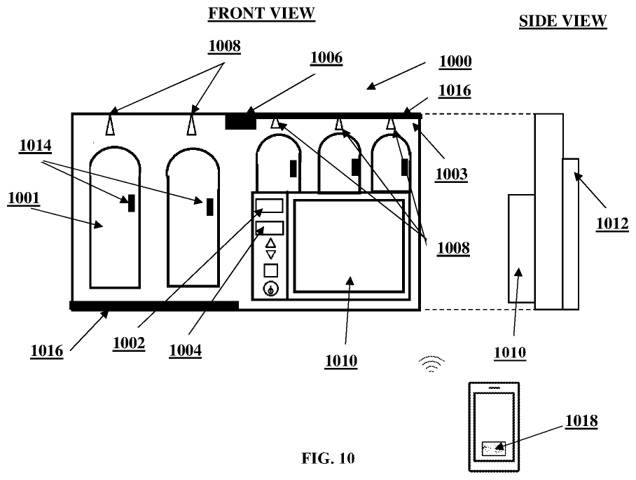

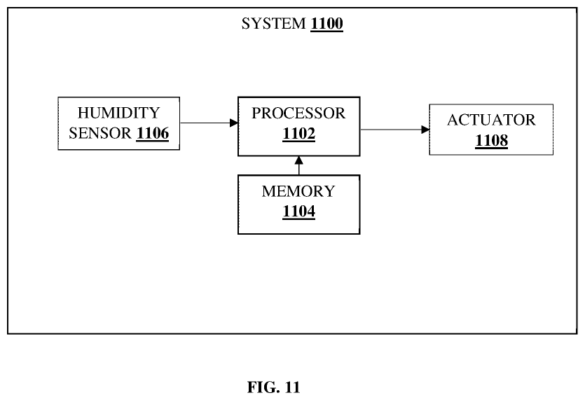

FIG. 10 shows a perspective view of an apparatus to control moisture according to an embodiment. The apparatus 1000 comprises a reed slot for holding reeds 1001, a processor 1002, a memory 1004, an actuator 1006, a spray 1008, a user interface 1010, a water compartment 1012; and humidity sensors 1014. The processor 1002 is communicatively coupled to the memory 1004. The spray 1008 comprises at least one of a jet spray, a mist spray, a dripper, and a humidifier. The apparatus 1000 comprises a storage case 1003 for one or more reeds of a musical instrument. The musical instrument may be a woodwind instrument. The humidity sensor, the spray, and the actuator may be located inside the storage case 1003 where the reeds are stored. The device may further comprise a first sensor in the storage case 1003 to monitor a fungus growth and a second sensor located on the reeds to monitor the damage of the reeds. The second sensors could be Ultrasound sensors that are fitted in the storage case used to analyze the quality and damage of the reeds using ultrasonic waves. In an embodiment, the second sensors could be strain gauges measure which measure deformation or strain on the reed. The storage case may further comprise an Ultraviolet (UV) sterilization system 1016 to sterilize the case and the reeds.

In an embodiment the user interface 1010 may be provided upon the storage box and may be detachable from the case. It can further be operated remotely. The user interface 1010 comprises at least one of an interactive display, or a handheld remote device. A mobile device may further function as a user interface providing interactive display. In an embodiment, control of the apparatus is through an application programming interface. The communication between the apparatus/device may be via a remote device and may be Bluetooth®, Wi-Fi® enabled. A dedicated smartphone application (App) 1018 can be used to remotely monitor and adjust the reed's moisture levels.

In an embodiment, the processor is operable to receive an instruction to actuate the actuator to activate the spray. The apparatus is operable to control humidity of a reed stored inside a storage. The instruction received is one or more of a time input, a time interval, and a quantity of water to be sprayed.

In an embodiment, the processor is configured to receive feedback of the humidity inside the apparatus through the humidity sensor. The processor is operable to receive the value of humidity, compare the value of humidity with a pre-set range of humidity values. The apparatus maintains the pre-set range of humidity in the reed. The pre-set value of the humidity is set through a user interface. Determining the preset value or range may be based on the ideal moisture level for reeds. It may be determined by the user based on a trial and observation, where the user adjust moisture levels to find a balance between good playability and avoiding damage. This entails experimenting with different moisture levels while considering factors such as the instrument type, reed material, environmental conditions, and feedback from experienced players. The ideal moisture level ensures a balanced, responsive sound without being too dry (resulting in a thin, harsh sound) or too wet (causing a dull, less controlled sound). Visual inspection, considering environmental factors, and data analytics on various moisture levels and user instrument playability contribute to finding the best moisture level. In an embodiment, the moisture level may be expressed as a range within which the humidity value can be maintained. The smartphone Application may collect data over time from the vibration measuring sensors fitted on or near the reeds to analyze reed playability and quality of sound and determine the ideal moisture level for each reed and that may be set as a pre-determined moisture level for the reed. If the storage case comprises multiple reeds and each of different brand or make, having different material, the processor is enabled to maintain individual level of moisture for each reed. For such cases each reed may be fitted with a dedicated sensor for knowing the humidity level on the reed and a dedicated spray to release specific amount of water for the reed. In an embodiment the spray can be moveable and rotatable to over more than one reed in the storage case.

In an embodiment, the processor is operable to generate an alert if the value of humidity is lower than the pre-set range of humidity in the reed. A user may remotely operate the apparatus to turn on the actuator or to turn off the actuator.

In an embodiment, the processor is configured to detect at least one of a fungus and moss growth on the reed through the first sensor. The processor is operable to generate an alert if fungus or moss growth is detected on the reed. In an embodiment, the water compartment may have water containing a disinfectant to avoid growth of fungus or moss on the reed. In an embodiment, the UV sterilization system 1016 may be activated when a fungus or moss growth is detected. In an embodiment, the water compartment may be fitted with a sensor or an indicator to alert the user when the water in the water compartment reaches to zero or a set lower threshold.

In an embodiment, the system is set according to the schedule of the user. The schedule of the user is the date and time of a performance in which a user may use the reed. The apparatus sprays water just before a pre-set time before the date and time of the performance of the use. This may avoid moistening of the reeds unnecessarily if the reeds are not in use. This also avoids the growth of fungus and moss on the reed. In an embodiment, the schedule of the user may be accessed via the user device by the apparatus. The apparatus regulates and monitors reed moisture levels. This system can help prevent over-soaking or drying out reeds, ensuring they remain in the optimal condition for playing. The humidification control module of the processor and the humidity measuring sensors are used to maintain the right moisture level.

In an embodiment, a small, lightweight sensor may be attached to the reed itself. This sensor measures moisture levels, temperature, and humidity in real time. It sends this data to a control unit wirelessly, which can be a smartphone app or a dedicated device. A control unit/module receives data from the humidity sensors 1014 and provides a user interface 1010 for musicians to monitor and adjust the reed's moisture level. The control unit regulates the moisture content of the reed. Depending on the readings from the sensor and user preferences, it can release small amounts of moisture or absorb excess moisture to maintain the ideal moisture level. A dedicated smartphone application (App) can be used to remotely monitor and adjust the reed's moisture levels. Musicians can set preferences for their desired moisture range and receive notifications when adjustments are needed. The apparatus is a Reed Moisture Management System is a smart device designed to monitor and control the moisture levels of woodwind reeds.

In an embodiment, the memory 1004 or the smartphone App may store the ideal moisture levels based on the manufacturer's instructions for the reed, and material of the reed.

In an embodiment, the processor is configured to detect at least one of a crack and a physical damage of the reed using a sensor that is attached to the reed and while the reed is being used. The processor is operable to generate an alert if the crack or physical damage of the reed is detected. Monitoring vibrations and potential damage in reeds is necessary for maintaining woodwind instrument quality and can be achieved using various sensors. Piezoelectric sensors, sensitive to vibrations, can be placed on or near the reed to detect minute changes during play. Accelerometers, whether piezoresistive, capacitive, or MEMS-based, attached to the instrument or reed, track motion and force applied during play. Strain gauges placed strategically on the reed measure bending, providing insight into stress and potential damage. Fiber optic sensors integrated into the reed structure can capture subtle changes in bending and vibrations. Acoustic sensors or microphones around the instrument, while not directly measuring reed vibrations, indirectly detect changes in sound. These sensors, integrated into a monitoring device, could wirelessly transmit data to a smartphone app or dedicated device, offering real-time feedback on the reed's condition, enabling informed decisions about maintenance, rotation, or replacement. The app may further provide real-time feedback on the reed's condition. It may track how many hours the reed has been used and offers recommendations based on its performance history. The app can also suggest reed rotation schedules to balance wear across multiple reeds.

FIG. 11 shows a perspective view of the system to control moisture in one embodiment. The system 1100 further comprises a processor 1102, a memory 1104, a humidity sensor 1106, and an actuator 1108. The processor 1102 is configured to control the moisture of the reed. The processor 1102 receives feedback from the humidity sensor 1106, that is a value of the moisture in the reed. The processor receives a value of humidity of the reed. The processor 1102 further compares the value of humidity with a pre-set range of humidity for the reed. If the value of humidity is more than the pre-set value, the processor 1102 turns on the actuator 1108 to turn on the spray. The spray sprays water onto the reed in the form of mist or humidifier. The water is drawn from the water compartment in the apparatus. The system 1100 further comprises a pump to draw water. This moistens the reed. Once an optimum level of the humidity is reached the spray is turned off. The optimum level of humidity is the pre-set value of humidity of the reed. The pre-set value of humidity is set using a user interface. The user interface is an interactive display, a mobile device, and a handheld remote device. The amount of water to be sprayed is decided by the number of reeds in the storage. A third sensor may keep a count of the number of reeds in the storage. Thus, the system maintains the pre-set range of humidity in the reed to keep the reed moist to avoid damage to the reed.

In an embodiment, the device comprises a sensor to detect if the reed of the instrument is cracked or damaged.

In an embodiment, control of the system is through an application programming interface. The communication between the system and the remote device may be Bluetooth®, Wi-Fi® enabled.

An embodiment relates to a method comprising receiving a first value of humidity of a reed; turning on an actuator; receiving a second value of humidity of the reed; turning off the actuator when the reed reaches a pre-set range of humidity; and the method is for controlling moisture of the reed.

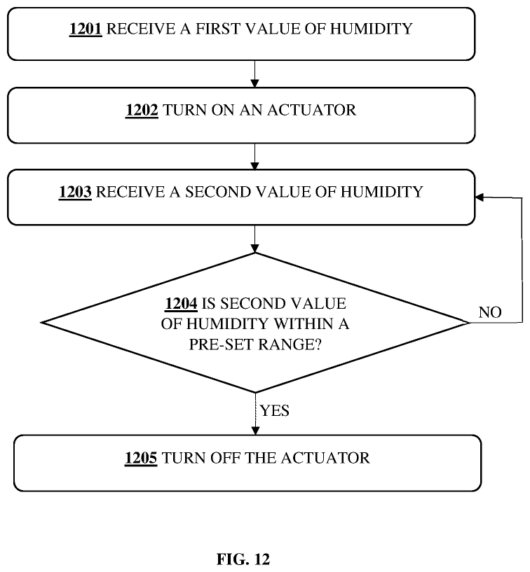

FIG. 12 shows a method for controlling moisture of the reed.

At step 1201, the first value of humidity of the reed is received through a humidity sensor. The first value of humidity is then compared with a pre-set range of humidity for the reed.

At step 1202, an actuator is turned on when the first value of humidity is less than the pre-set range of humidity.

At step 1203, the second value of humidity of the reed is received through a humidity sensor.

At step 1204, the second value of humidity is then compared with a pre-set range of humidity for the reed.

At step 1205, the actuator is turned off when the second value of humidity is within the pre-set range of humidity.

According to an embodiment, it is a method comprising determining a first value of humidity of a reed via a humidity sensor; turning on an actuator operable to spray a water mist on the reed; determining a second value of humidity of the reed; turning off the actuator when the reed reaches a pre-set value of humidity; and wherein the method is for controlling moisture of the reed stored in a storage case.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

### INCORPORATION BY REFERENCE

All references, including granted patents and patent application publications, referred to herein are incorporated herein by reference in their entirety.
U.S. Publication Number 20180289869 titled “Dental device and suction device”
U.S. Publication Number 20070221039 titled “Moisture cap assembly for reed instruments”.
U.S. patent Ser. No. 10/832,641 titled “Humidity controlled musical instrument reed cap.”
U.S. Publication Number 20180289869 titled “Dental device and suction device”
U.S. Publication Number 20070221039 titled “Moisture cap assembly for reed instruments”.
U.S. patent Ser. No. 10/832,641 titled “Humidity controlled musical instrument reed cap.”

What is claimed is:

1. An apparatus comprising:
a spray;
an actuator;
a water compartment;
a memory; and
a processor; wherein the processor is communicatively coupled to the memory and wherein the processor is operable to:
actuate the actuator to activate the spray; and
wherein the apparatus is operable to control humidity of a reed stored in a storage case.

2. The apparatus of claim 1, wherein the processor is operable to receive an instruction to actuate the actuator.

3. The apparatus of claim 1, wherein the spray comprises at least one of a jet spray, a mist spray, a dripper, and a humidifier.

4. The apparatus of claim 1, wherein the apparatus comprises a humidity sensor to measure a value of humidity of the reed inside the storage case.

5. The apparatus of claim 1, wherein the processor is configured to automatically switch on the spray based on a user schedule accessed via an application.

6. The apparatus of claim 1, wherein the processor is operable to:
receive a value of humidity;
compare the value of humidity with a pre-set value of humidity; and
wherein the apparatus maintains a moisture in the reed in the pre-set value of humidity.

7. The apparatus of claim 1, wherein the processor is operable to generate an alert if a value of humidity is lower than a pre-set value of humidity in the reed.

8. The apparatus of claim 7, wherein the apparatus is controlled remotely to turn on the actuator.

9. The apparatus of claim 1, wherein the apparatus further comprises a first sensor to detect at least one of a fungus and moss growth on the reed.

10. The apparatus of claim 1, wherein the apparatus further comprises a UV sterilization unit to sterilize the reed and the storage case.

11. The apparatus of claim 1, wherein the apparatus further comprises a second sensor to detect at least one of a crack and a physical damage of the reed.

12. The apparatus of claim 7, wherein the pre-set value of the humidity is set through a user interface or via a smartphone Application.

13. The apparatus of claim 1, wherein the user interface comprises at least one of an interactive display, a mobile device, and a handheld remote device.

14. The apparatus of claim 1, wherein the control of the apparatus is through a smartphone Application.

15. A system comprising:
a storage case comprising,
a spray;
an actuator;
a water compartment;
a memory; and
a processor;
an application stored on a user device;
wherein the processor is communicatively coupled to the memory and wherein the processor is operable to activate the spray via the actuator via the application stored on the user device; and
wherein the system is operable to control humidity of a reed stored in the storage case.

16. The system of claim 15, wherein the instruction received is at least one of a time input, a time interval, and a quantity of water to be sprayed.

17. The system of claim 15, wherein the spray comprises at least one of a jet spray, a mist spray, a dripper, and a humidifier.

18. The system of claim 15, wherein the system comprises a humidity sensor to measure a value of humidity of the reed.

19. The system of claim 15, wherein the processor is operable to:
receive the value of humidity;
compare the value of humidity with a pre-set value of humidity; and
wherein the system maintains humidity in the reed in the pre-set value.

20. A method comprising:
determining a first value of humidity of a reed via a humidity sensor;
turning on an actuator operable to spray a water mist on the reed;
determining a second value of humidity of the reed;
turning off the actuator when the reed reaches a pre-set value of humidity; and
wherein the method is for controlling moisture of the reed stored in a storage case.
